(19) United States
(12) Patent Application Publication
(10) Pub. No.: US 2014/0163327 A1
(43) Pub. Date: Jun. 12, 2014

(54) METHOD AND SYSTEM FOR REMOTE THYROIDECTOMY AND PARATHYROIDECTOMY

(71) Applicant: BOSTON SCIENTIFIC SCIMED, INC., Maple Grove, MN (US)

(72) Inventor: Lee L. SWANSTROM, Portland, OR (US)

(73) Assignee: BOSTON SCIENTIFIC SCIMED, INC, Maple Grove, MN (US)

(21) Appl. No.: 14/103,261

(22) Filed: Dec. 11, 2013

Related U.S. Application Data
(60) Provisional application No. 61/736,117, filed on Dec. 12, 2012.

(51) Int. Cl. A61B 17/32; A61B 17/02

(52) U.S. Cl. CPC A61B 17/320016; A61B 17/0218
USPC 600/235; 606/170

(57) ABSTRACT

Embodiments include a method of performing a medical procedure in a patient. The method includes creating an incision in the patient and inserting a flexible guide tube into the incision. The method also includes locating a flexible instrument and a flexible optical device within the guide tube, tunneling the distal tip of the guide tube through the incision to a sternal notch or a position proximate the sternal notch, and dissecting at least part of a thyroid using the instrument.

### CROSS-REFERENCE TO RELATED APPLICATIONS

This application claims the benefit of priority from U.S. Provisional Application No. 61/736,117, filed on Dec. 12, 2012, the entirety of which is incorporated by reference herein.

### FIELD

Embodiments of the present invention generally relate to medical devices. Specifically, embodiments of the present invention relate to systems and devices for use during surgical procedures, such as, a thyroidectomy. Embodiments of the present invention also encompass related procedures.

### BACKGROUND

It is generally desirable to minimize the invasiveness of medical procedures, including therapeutic or diagnostic procedures. Invasive “open” medical procedures are generally more expensive than minimally invasive “keyhole” procedures, and there is often a greater risk of complication and discomfort for the patient. Since open surgery typically requires relatively large incisions, blood loss can be high and the risk of infection or other post-operative complications may increase. Large incisions may require extended recovery times to heal and leave unsightly scarring. Internal and external scar tissue from open surgery can cause lifetime morbidities such as pain, hernias and bowel obstructions. Accordingly, methods, systems, and devices that reduce trauma to the patient, are less invasive, or enhance recovery are desirable.

A thyroidectomy is a surgical procedure whereby part or all of the thyroid gland is removed. Laparoscopic and robotic thyroidectomies have been developed that use rigid devices inserted near the axilla. A transaxillary approach has the advantage of leaving no cervical scarring, but suffers from several drawbacks. Rigid devices, laparoscopic or robotic, must be pivoted about the incision through which they are passed. Precise control of the distal end of a rigid device can be difficult due to this pivoting movement. One way to compensate for the “pivoting” phenomenon is to enlarge the access incision. Many current minimally invasive approaches result in larger overall incisions compared to open approaches. Visualization may also be complicated because a separate optical device is required. In addition to possibly requiring an extra incision, the optical device must be appropriately positioned to provide a useful field of view of the surgical site. Maintaining or adjusting the optical device relative to the rigid device is time consuming and may involve constant attention. Moreover, the transaxillary approach provides limited access to an ipsilateral (i.e., proximal) lobe, and even less access to a contralateral (i.e., distal) lobe.

Standard endoscopic devices are also unsuitable for thyroidectomies, in part, because the approach is not through a natural body cavity. Standard endoscopes are specifically designed to flex to pass through the lower gastrointestinal tract or the esophagus. Consequently, they are not configured for use in procedures requiring navigation past organs or through tissue without surrounding support structure to guide them. Standard endoscopic devices are too long or too flexible for such navigation. In addition, these devices are not configured for ergonomic control of instruments passed through them. Control of a standard endoscopic device often requires two hands to operate, meaning that only one device can be operated at a time by a surgeon. Nor can standard endoscopic devices be locked or moved incrementally relative to a patient. The present disclosure overcomes at least some of the limitations of prior art.

It is to be understood that both the foregoing general description and the following detailed description are exemplary and explanatory only and are not restrictive of the invention.

### SUMMARY

In one aspect, a method of performing a medical procedure in a patient includes creating an incision in the patient, and inserting a flexible guide tube into the incision. The method also includes locating a flexible instrument and a flexible optical device within the guide tube, tunneling the distal tip of the guide tube through the incision to a sternal notch or a position proximate the sternal notch, and dissecting at least part of a thyroid using the instrument.

In another aspect, a method of performing a medical procedure in a patient includes creating a sub-xiphoid incision in the patient, passing a distal tip of a flexible guide tube through the incision, and positioning a distal tip of the guide tube in a lower subcutaneous location. The method also includes tunneling the distal tip of the guide tube from the lower subcutaneous location to an upper subcutaneous location, advancing a first flexible instrument and a second flexible instrument through the guide tube, and dissecting at least part of a thyroid using the first instrument and the second instrument.

In a further aspect, a method of performing a medical procedure in a patient includes creating an incision in an umbilicus of the patient, passing a distal end of a flexible guide tube and a distal end of a flexible instrument through the incision, and creating a pathway from the incision to a thyroid using at least one of the guide tube and the instrument, wherein a part of the pathway is subcutaneous. The method also includes positioning an optical device to visualize the thyroid and dissecting at least part of the thyroid from surrounding tissue using the instrument.

Additional objects and advantages of the invention will be set forth in part in the description which follows, and in part will be obvious from the description, or may be learned by practice of the invention. The objects and advantages of the invention will be realized and attained by means of the elements and combinations particularly pointed out below.

It is to be understood that both the foregoing general description and the following detailed description are exemplary and explanatory only and are not restrictive of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A is a perspective view of an endoscopic system, according to an exemplary embodiment;

FIG. 1B is an enlarged perspective view of a distal region of the endoscopic system shown in FIG. 1A, according to an exemplary embodiment;

FIG. 2 is a perspective view of a guide tube, according to an exemplary embodiment;

FIG. 3 is a perspective view of an instrument, according to an exemplary embodiment;

FIG. 4A is a side view of an instrument end effector showing articulation in three different positions, according to an exemplary embodiment;

FIG. 4B is a side view of five instrument end effectors, according to exemplary embodiments;

FIG. 5 is a perspective view of a platform, according to an exemplary embodiment;

FIG. 6 is an exploded view of the platform shown in FIG. 5, according to an exemplary embodiment;

FIG. 7A is a perspective view of a slide assembly, according to an exemplary embodiment;

FIG. 7B is an exploded view of the slide assembly shown in FIG. 7A, according to an exemplary embodiment;

FIG. 8 is a schematic representation of some cervical organs of a patient from an anterior perspective;

FIG. 9 is a schematic representation of some cervical organs of a patient from a posterior perspective;

FIG. 10 is a schematic representation of some thoracic organs of a patient;

FIG. 11A is a flow chart of a medical procedure, according to an exemplary embodiment; and

FIG. 11B is a flow chart of a medical procedure, according to an exemplary embodiment.

### DESCRIPTION OF THE EMBODIMENTS

Reference will now be made in detail to exemplary embodiments of the invention, examples of which are illustrated in the accompanying drawings. Wherever possible, the same reference numbers will be used throughout the drawings to refer to the same or like parts.

The terms “proximal” and “distal” are used herein to refer to the relative positions of the components of the exemplary endoscopic system 10. When used herein, “proximal” refers to a position relatively closer to the exterior of the body or closer to the surgeon using endoscopic system 10. In contrast, “distal” refers to a position relatively further away from the surgeon using endoscopic system 10 or closer to the interior of the body.

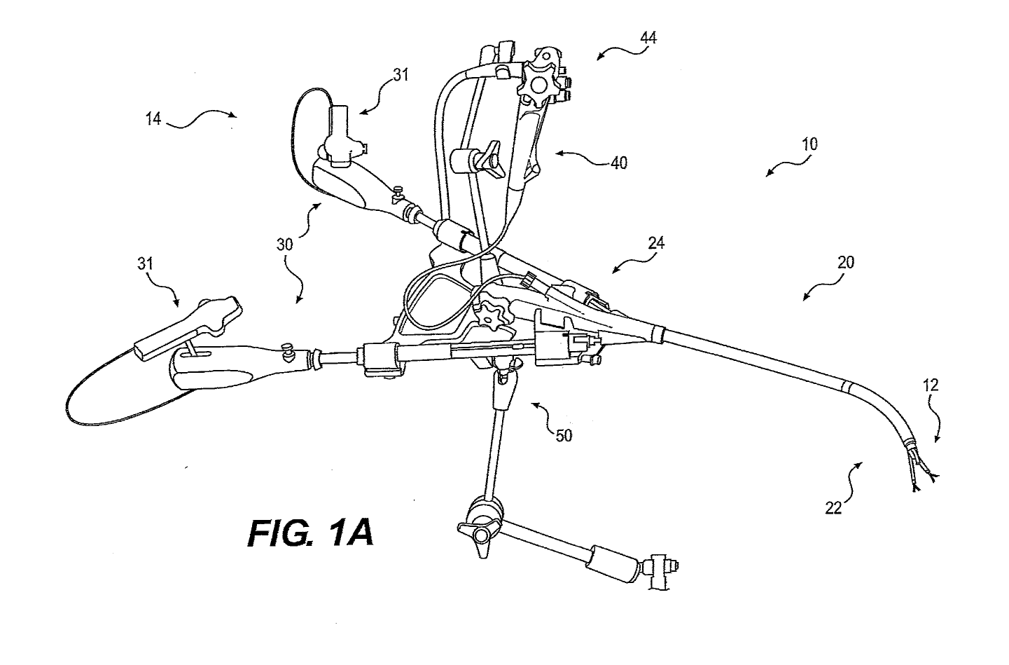

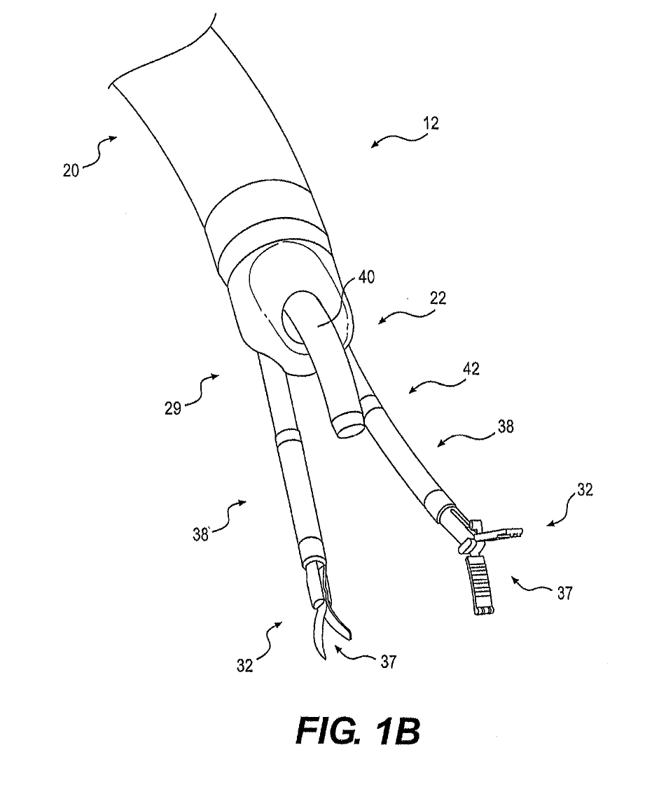

FIG. 1A depicts an endoscopic system 10, according to an exemplary embodiment. Endoscopic system 10 can include a direct drive endoscopic system (DDES), and may be used for a therapeutic or a diagnostic procedure. Such procedures may be performed by inserting an endoscopic device, guide tube, catheter, or other medical device into the body through an anatomic opening (e.g., an incision or a natural orifice).

Although in the description that follows, endoscopic system 10 is described and shown as being inserted into the body through an incision in a patient, it should be emphasized that this description is exemplary only. For example, endoscopic system 10 may also be used for procedures in or near other body organs, such as trachea, larynx, or other cervical organs. Embodiments of the current disclosure may be used in, but are not limited to, single incision laparoscopic surgical (SILS) procedures, complex endoluminal surgeries or natural orifice transluminal endoscopic surgery (NOTES) procedures.

According to an exemplary embodiment, endoscopic system 10 may include a guide tube 20, one or more instruments 30, an optical device 40, and a platform 50. A distal region 12 of endoscopic system 10 may be inserted into a patient to perform various medical procedures as described herein. During these procedures, a proximal region 14 of endoscopic system 10 remains outside the patient and is manipulated by a surgeon to control components of distal region 12, as shown in FIG. 1B.

Endoscopic system 10 is configured to permit a surgeon to operate two instruments 30 simultaneously. In other embodiments, the surgeon may operate one instrument 30 while moving platform 50 or adjusting optical device 40. Generally, platform 50 and guide tube 20 are configured to permit simultaneous and independent operation of two instruments 30 using one hand on each instrument 30. Platform 50 can be rigidly coupled to an operating table, and thus be fixed relative to a patient during an operation. Platform 50 can also be locked and unlocked to permit movement relative to the operating table or patient.

Guide tube 20 and optical device 40 can be removably coupled to platform 50. Optical device 40 can include a commercially available endoscope or other imaging device. Other features and operations of the various components of endoscopic system 10 will be described below. Some exemplary embodiments of guide tube 20, instrument 30, optical device 40, and platform 50 (frame) are disclosed, for example, in U.S. patent application Ser. No. 11/946,790, entitled “Multi-Part Instrument Systems and Methods,” which is hereby incorporated by reference in its entirety.

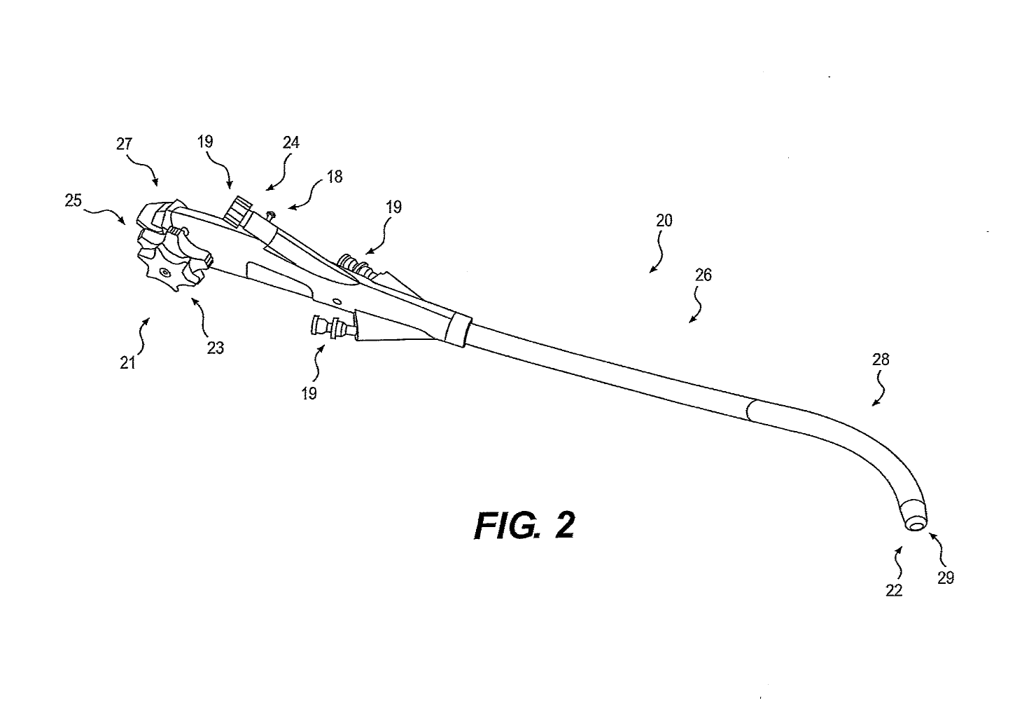

As shown in FIG. 2, guide tube 20 may include a proximal region 24 housing one or more ports 19 configured to receive one or more instruments 30 or optical devices 40. Lumens (not shown) may extend longitudinally (axially) between ports 19 and a distal region 22 of guide tube 20. A port 18 can be configured to receive an insufflation gas, wherein port 18 is fluidly connected to distal end 22. In other embodiments, guide tube 20 can include integrated optics (not shown), such as, for example, a light source, wave guide, imaging chip, or other similar components.

Guide tube 20 may include a region 26 and an articulating region 28. Region 26 can be flexible enough to bend around organs yet rigid enough to adequately transfer compressive force from proximal region 24 to distal region 22. For example, region 26 could be configured to bend to allow distal region 22 to be navigated from an incision in the umbilicus to an inferior surface of the diaphragm. Such navigation requires sufficient flexibility to flex in order to pass around organs in the abdominal cavity and sufficient rigidity to force distal region 22 through connective tissue supporting the abdominal organs.

Articulating region 28 may be controllably moved via a control mechanism 21 located at proximal region 24. For example, a first knob 23 could be rotated to move articulating region 28 left and right while a second knob 25 could be rotated to move articulating region 28 up and down. A brake 27 can be configured to lock or unlock the movement of articulating region 28. Controlled movement of articulating region 28 can control movement of distal region 22 of guide tube 20, a distal region 32 of instrument 30, or a distal region 42 of optical device 40 relative to a patient.

FIG. 1B shows distal region 12 of endoscopic system 10, according to an exemplary embodiment. Specifically, distal region 42 of optical device 40 is shown extended from distal region 22 of guide tube 20. Likewise, two instruments 30 are shown with their distal ends 32 extended from distal region 22. One or more instruments 30 or optical device 40 may be advanced from, or retracted into, distal region 22. Distal region 22 can include a rounded distal tip 29 configured to aid passage of guide tube 20 through tissue.

Optical device 40 can be moved relative to guide tube 20 to adjust a viewing angle, viewing distance, or location of distal end 42 relative to the patient or the surgical site. In some embodiments, an articulating region (not shown) can be configured to permit movement and repositioning of distal end 42 via movement of various controls located at a proximal region 44 of optical device 40 (FIG. 1A). Optical device 40 may be independently moveable with respect to guide tube 20 or instrument 30. For example, optical device 40 may be moveably received in a lumen in guide tube 20, as shown in FIG. 1B. The surgeon may move optical device 40 longitudinally, laterally, or rotationally to control the position of distal end 42 with respect to guide tube 20. Distal end 42 of optical device 40 may also be positioned with respect to instrument 30 so that optical device 40 provides a “bird's eye view” of the surgical site or end effectors 37 of instruments 30.

In some embodiments, optical device 40 may be embedded into or attached to guide tube 20 or instrument 30. For example, optical device 40 may be on a distal-facing, circumferential, or other outer surface near distal ends 22, 32. With such an “integral” optical device 40, the surgeon may move guide tube 20 or instrument 30 longitudinally, laterally, or rotationally to control the position of optical device 40 with respect to the patient.

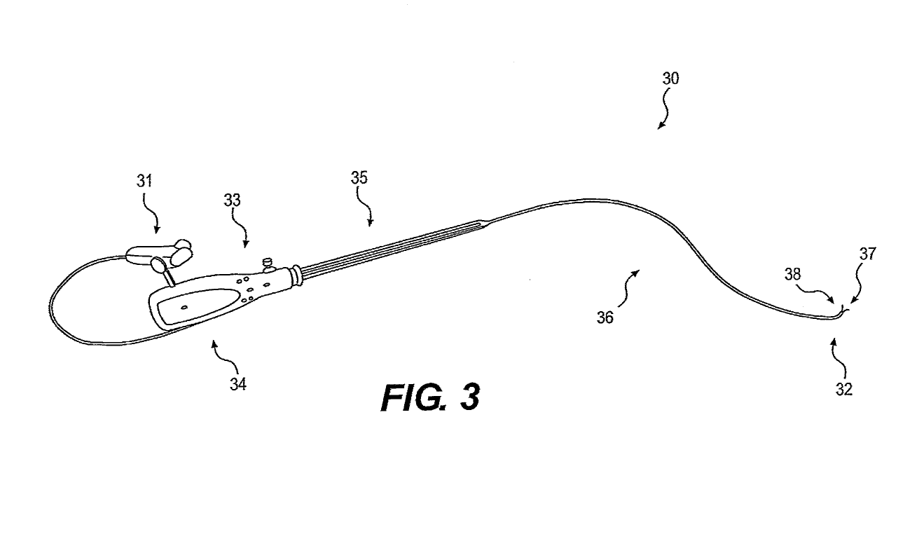

In use, distal end 32 of instrument 30 may be articulated by moving articulating region 38. For example, actuators 31 (shown in FIGS. 1A, 3) may be moved relative to instruments 30 to control up/down and left/right movement of articulating regions 38 similar to that described above for articulating region 28. The surgeon may move each instrument 30 longitudinally (e.g., in the distal and proximal directions, axially), laterally (e.g., side to side or up and down), or rotationally with respect to guide tube 20.

FIG. 3 shows a perspective view of instrument 30, according to an exemplary embodiment. Instrument 30 includes a proximal region 34 moveably coupled to actuator 31. Actuator 31 can be rotatably coupled to a controller body 33 such that rotation about two or more axes can control movement of articulating region 38. For example, actuator 31 can be rotated clockwise or anticlockwise to move articulating region 38 left and right. Actuator 31 can also be tilted forward or backwards relative to controller body 33 to move articulating region 38 up or down. Some exemplary embodiments of actuator 31 are disclosed, for example, in U.S. Pat. No. 8,057,462, entitled “Medical Device Control System,” which is hereby incorporated by reference in its entirety.

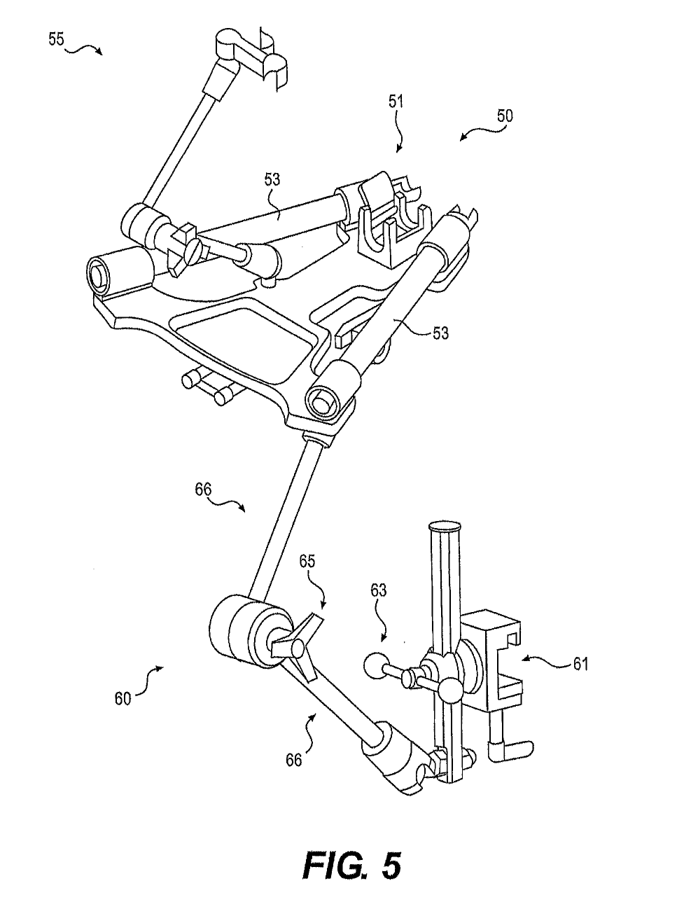

Instrument 30 can also include rigid region 35 configured for placement in a bearing tube 53 (FIG. 5), as described below. Region 35 does not generally extend into guide tube 20, but is rather used as a bearing to support instrument 30 on platform 50. Instrument 30 also includes a flexible region 36 configured for placement within guide tube 20. Similar to guide tube 20, the structural properties of region 36 can also be varied to provide sufficient flexibility to maneuver around organs and sufficient rigidity to exert a dissecting force on tissue.

Each instrument 30 can also include end effector 37 located at distal region 32. Instrument 30 may include one or more control elements (not shown) connected to end effector 37 to allow the surgeon to control the movement of end effector 37. End effector 37 may be connected to articulating region 38 to allow end effector 37 to move up/down or left/right. Instrument 30 and end effector 37 may include a range of configurations for use with various medical procedures.

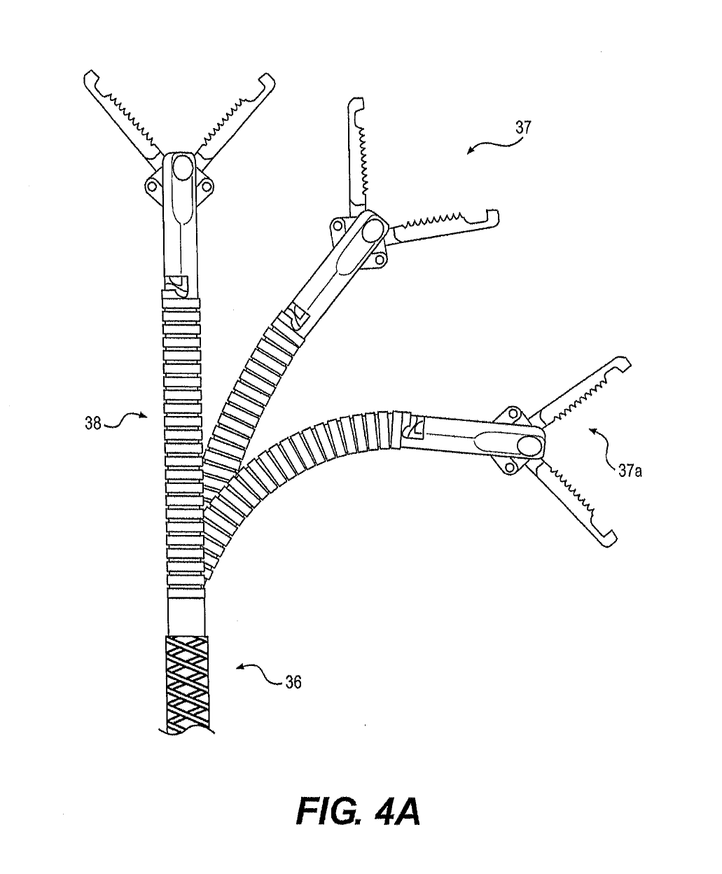

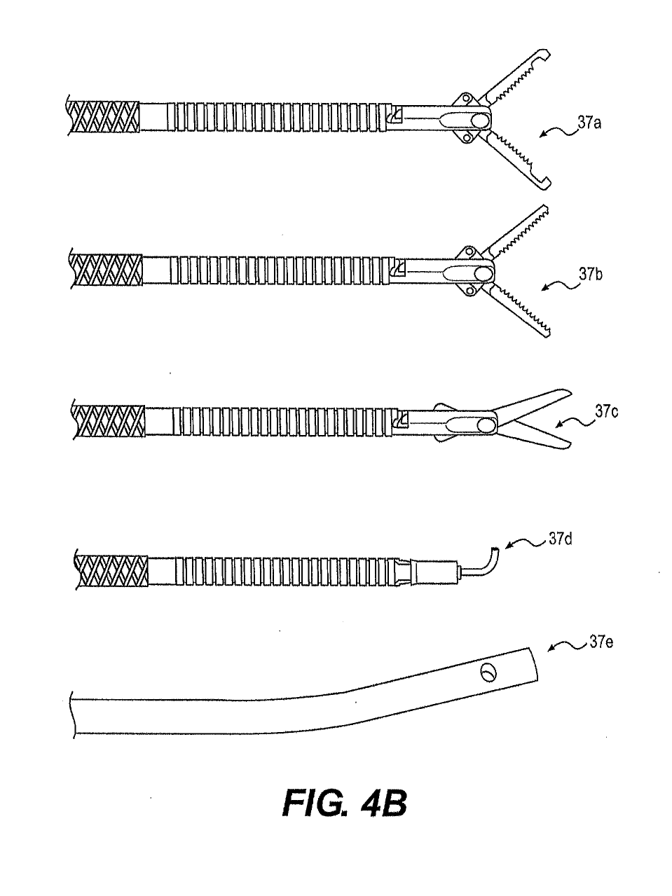

FIGS. 4A, 4B show various end effectors 37 that may form part of instrument 30. End effector 37 may include a device configured to assist in performing a surgical procedure. FIG. 4A shows the movement of articulating region 38, including a grasper end effector 37a. Various other types of end effectors 37 are contemplated for use with instrument 30. Generally, end effector 37 may include, but is not limited to, a cutting device (e.g., scissors, tissue cutter, etc.), forceps, a fixation device, a manipulation device, a dissection device, a support device, a sealing device, a needle holder, a closure device (e.g., clips, staples, loops, ligator, suturing device, etc.), a retrieval device (e.g., snare, basket, loop, a fluid extraction device, etc.), a tissue exploration device (e.g., optical device, illumination device, etc.), a tissue sampling device, a delivery device, a device for aiding in the patency of a lumen or for dilating an opening (e.g., a balloon or other expandable member, patency brush, stent, fan retractor, wire structure, etc.), a grasping device, a stabilizing device, an ablation device, a resection device, a pressure application device, an energy delivery device, etc. Instrument 30 may include a blunt or rounded tip for exploration or for assisting another instrument 30 or end effector 37 (e.g., an obturator). For example, as shown in FIG. 4B, grasper 37a, a dissector 37b, a pair of scissors 37c, an electrosurgical device 37d, and a suction/irrigation tube 37e may be incorporated with instrument 30.

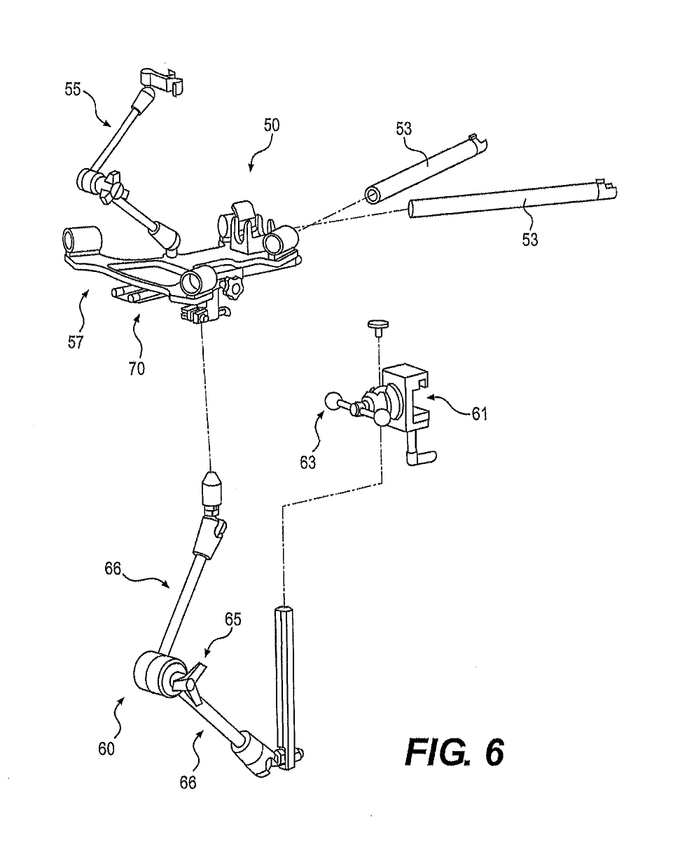

FIGS. 5 and 6 illustrate platform 50, according to an exemplary embodiment. As explained above, platform 50 can be attached to an operating table and configured to receive guide tube 20, one or more instruments 30, or optical device 40. Guide tube 20 can be fixedly mounted to platform 50 using a latch 51 or other attachment mechanism. Instrument 30 can be moveably coupled to platform 50 via bearing tube 53. Some exemplary embodiments of bearing tube 53 are disclosed, for example, in U.S. patent application Ser. No. 13/297,675, entitled “Bearing Assembly for Instrument,” which is hereby incorporated by reference in its entirety. Optical device 40 can be moveably coupled to platform 50 via an arm 55.

Platform 50 can be mounted to an operating table (not shown) using a mounting system 60. Mounting system 60 can include a clamp 61 configured to releasably couple to the operating table. A lever 63 may mechanically engage clamp 61 to provide locking/unlocking of select movement between mounting system 60 and clamp 61. In other words, locking lever 63 would limit movement and unlocking lever 63 would permit free movement of platform 60 in one or more directions. Alternatively, lever 63 could be moved to incrementally move (e.g., raise or lower) mounting system 60, and hence endoscopic system 10, relative to the operating table.

In addition to, or instead of, controlling movement using lever 63, a knob 65 may mechanically engage one or more mounting arms 66 to lock or free movement between two or more mounting arms 66. Alternatively, knob 65 could be moved to incrementally move two or more mounting arms 66 relative to each other.

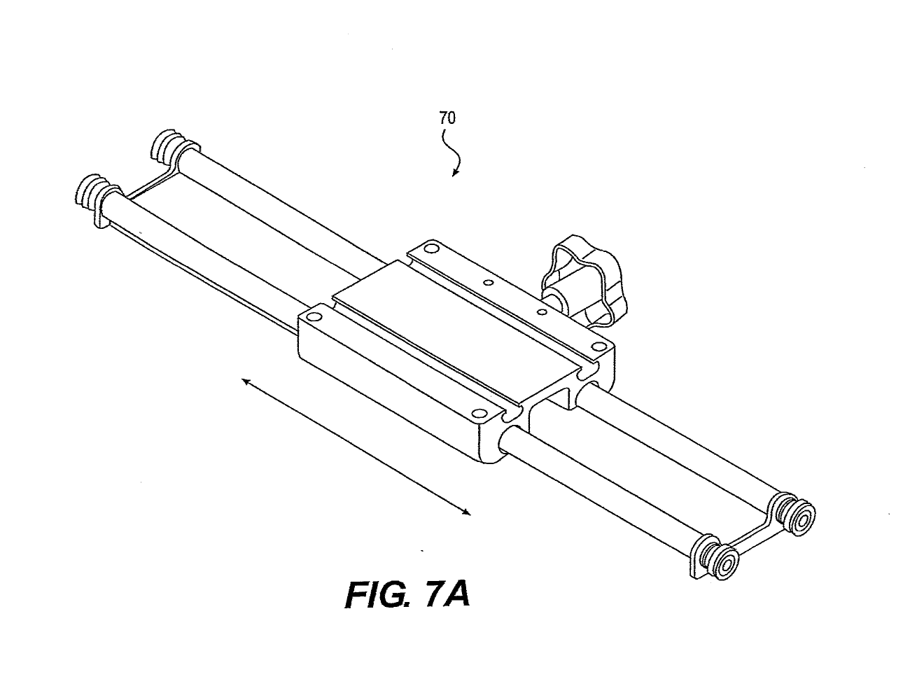

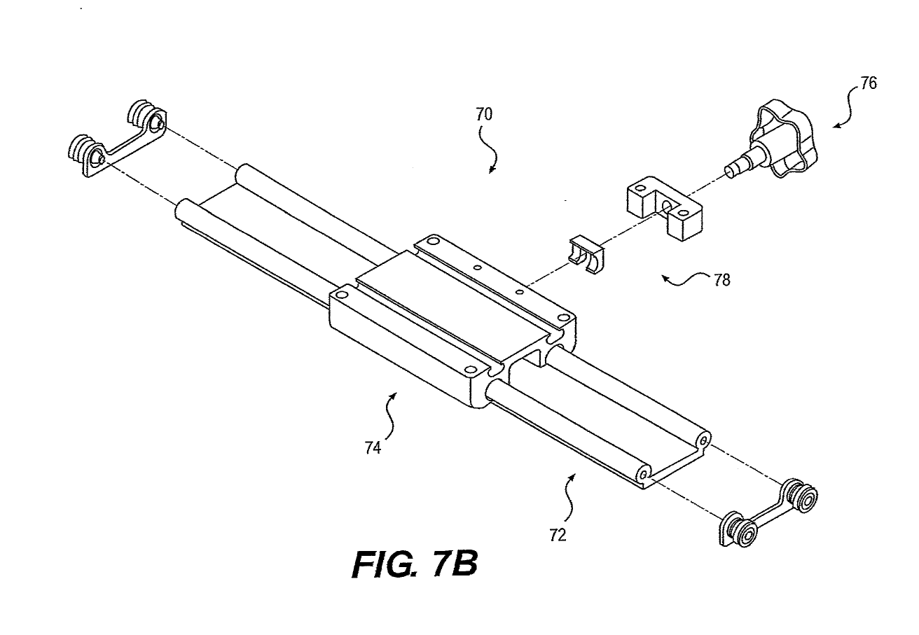

FIGS. 7A and 7B show a slide assembly 70, according to an exemplary embodiment. At least part of slide assembly 70 can be configured to move in one or more directions, as indicated by the double arrow in FIG. 7A. Slide assembly 70 can be located between mounting system 60 and a plate 57 of platform 50 (FIG. 6). Slide assembly 70 can include one or more rails 72 and a carriage 74 moveably coupled to the one or more rails 72. Plate 57 can be fixedly coupled to one or more rails 72 and carriage 74 can be fixedly coupled to mounting system 60. To control movement of carriage 74 relative to rails 72, slide assembly 70 can include a knob 76 and a mount 78 configured to couple knob 76 to carriage 74 or rails 72.

In use, knob 76 may mechanically engage carriage 74 or rails 72 to provide locking/unlocking of movement between carriage 74 and rails 72. Alternatively, knob 76 could be moved to incrementally translate carriage 74 relative to rails 72. As explained above with respect to mounting system 60, slide assembly 70 can be used to selectively move guide tube 20 relative to the operating table.

Various combinations of one or more levers 63, knobs 65, 76 or other devices could be used to provide select movement of endoscopic system 10 relative to the patient. In other embodiments, endoscopic system 10 can include a gear, belt, pulley, or other type of drive system configured to provide selective movement. For example, various components of endoscopic system 10 could be configured for locking/unlocking, incremental, or other types of select movement. Select movement of one or more components of endoscopic system 10 can provide advantages to a surgeon performing a procedure on a patient, as described herein.

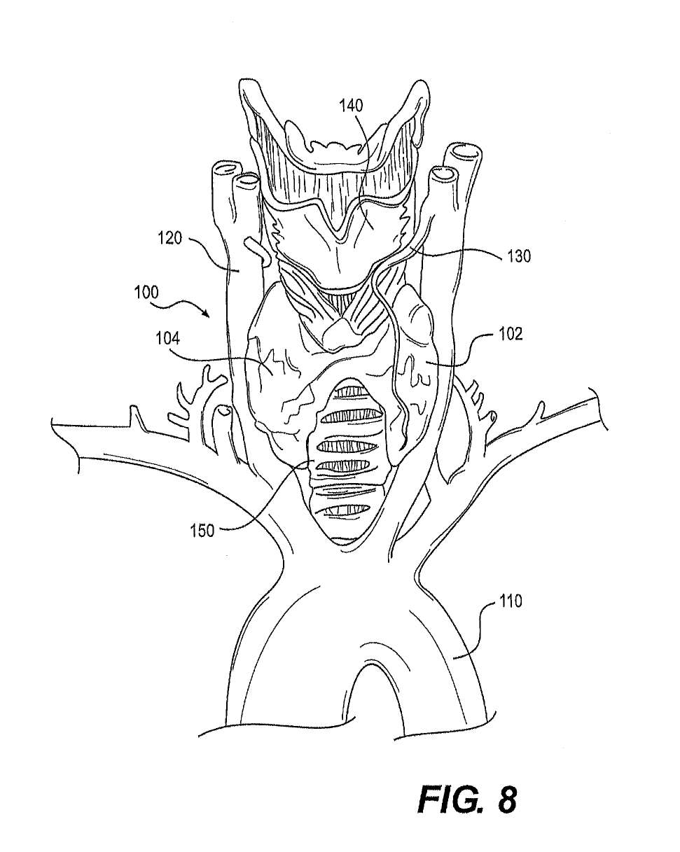

FIG. 8 shows a schematic illustration of some cervical organs of a patient from an anterior perspective. A thyroid 100 is shown surrounded by select vasculature, including an aorta 110, a common carotid artery 120, and a superior thyroid artery 130. Above thyroid 100 is a thyroid cartilage 140, and a trachea 150 is shown below. A left lobe 102, a right lobe 104, and an isthmus 106 of thyroid 100 are also shown.

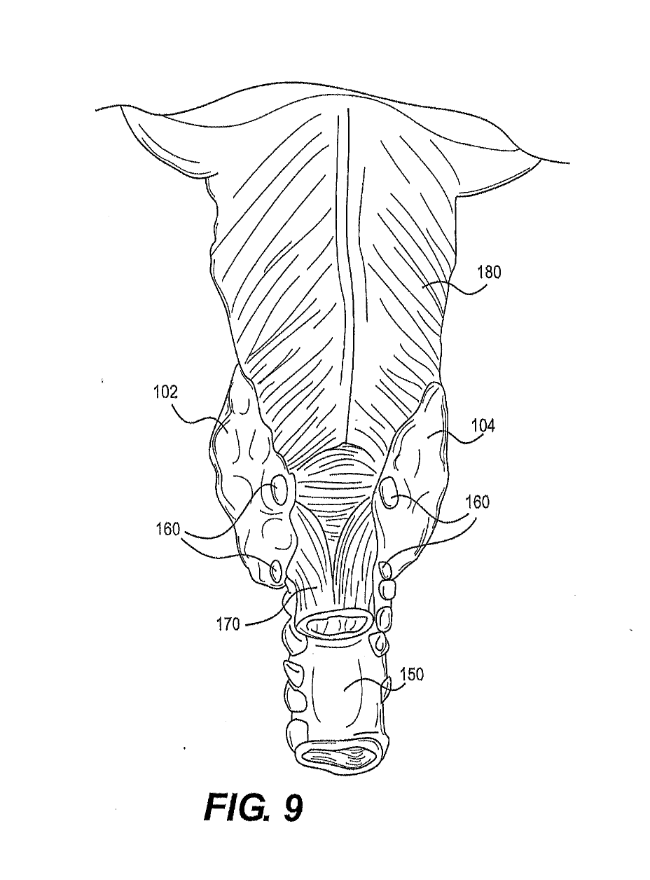

FIG. 9 shows a schematic illustration of thyroid 100 from a posterior perspective. Various parathyroid glands 160 are shown located on posterior surfaces of left lobe 102 and right lobe 104. Also shown are an esophagus 170 and a pharynx 180.

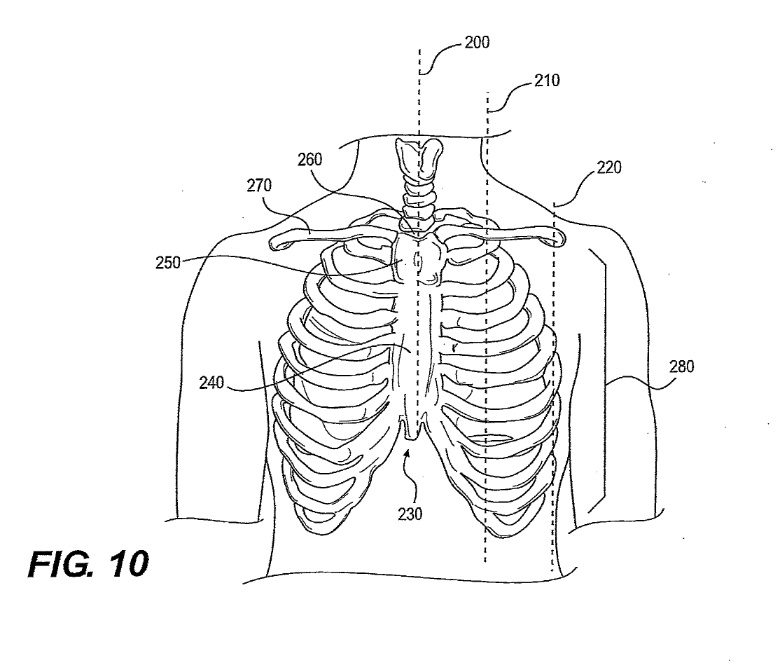

FIG. 10 shows a schematic illustration of a thoracic region of a patient. A midsternal line 200, a midclavicular line 210, and an anterior axillary line 220 are shown relative to a xiphoid 230, a sternum 240, a manubrium 250, and a sternal notch 260. Also shown are a clavicle 270 and various ribs 280.

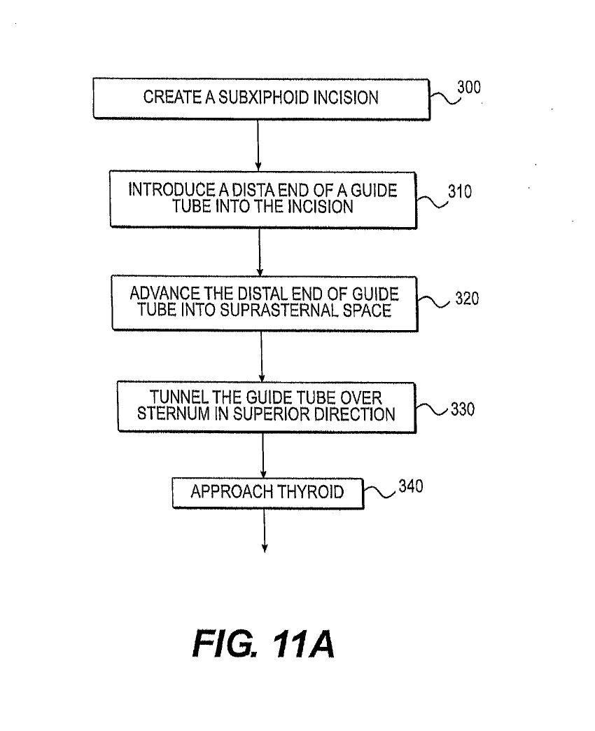

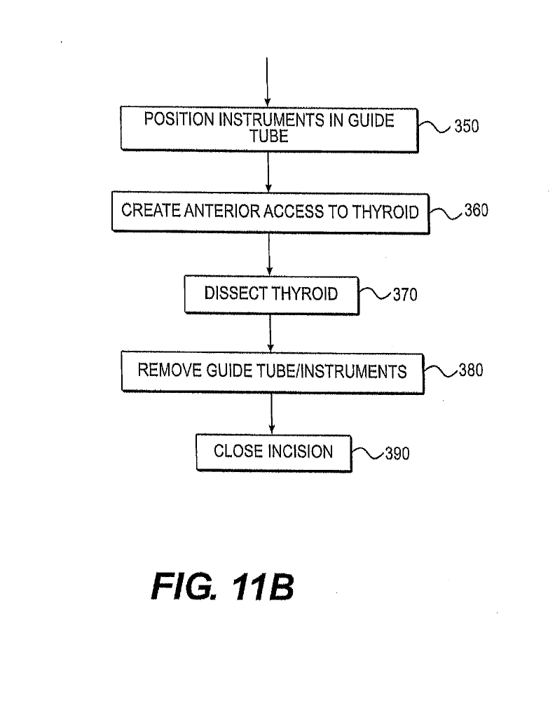

In accordance with an exemplary embodiment, a medical procedure may be performed as illustrated in the flow charts of FIGS. 11A, 11B. One or more of the steps shown or described herein may be omitted, modified, or repeated as necessary. For example, endoscopic system 10 could be used to perform the medical procedure shown in FIGS. 11A, 11B, or variations thereof. In another embodiment, endoscopic system 10 can be used to provide general access to cervical locations via a suprasternal approach.

In some embodiments, a single incision can be created (Step 300). The incision may be formed in an abdominal wall, and more specifically, through an anterior surface of the abdominal wall. The incision may be sub-xiphoid, including, for example, through the umbilicus. The incision may also be made between left and right midclavicular lines 210. The purpose of the incision is to permit subcutaneous access to a region located anterior to sternum 240.

Distal region 22 of guide tube 20 can be inserted through the incision (Step 310). Guide tube 20, instrument 30, and optical device 40 may be configured to be at least partially inserted into an anatomic opening. Guide tube 20, instrument 30, and optical device 40 may be advanced together through tissue. Guide tube 20, instrument 30, and optical device 40 may also be advanced separately in various combinations as each component can be moved independent of movement of one or more other components. For example, guide tube 20 may be advanced along with optical device 40 while one or more instruments 30 remain fixed relative to the patient.

During a procedure, one or more instruments 30 or optical device 40 can be readily removed from guide tube 20 and quickly replaced with another or a different component. The ability to precisely and quickly reposition a component at the surgical site allows a surgeon to quickly respond to unexpected events. For example, grasper 37a and dissector 37b can be quickly replaced with electrocautery hook 37d and suction tube 37e to treat a ruptured blood vessel.

To create and navigate a pathway to thyroid 100, various combinations of instrument 30, optical device 40, and guide tube 20 may be used to dissect tissue. For example, distal end 29 of guide tube 20 may be rounded and configured to deflect bone structures or separate tissue. Optical device 40 could also be used in some capacity to dissect or retract tissue. One or more instruments 30 can be configured to cut, dissect, remove, or ablate tissue. For example, a dissector, scissors, or an electrocautery hook may be used to, respectively, dissect, cut, or ablate tissue. Various components may be used to advance distal end 29 of guide tube 20 to a subcutaneous or suprasternal space (Step 320), as explained below. These techniques could also be used to tunnel guide tube 20, or another component, over sternum 240 in a superior direction (Step 330).

In some embodiments, guide tube 20 can be selectively moved relative to the patient to retract or dissect tissue. Select movement can include moving guide tube 20 relative to platform 50 over relatively small distances, such as, for example, less than one inch. Moving platform 50 relative to the patient can include relatively large distance, such as, for example, advancing guide tube 20 several inches.

Movement over different relative scales can be combined with locked, free, incremental, or other types of select movement to provide improved retraction or dissection using endoscopic system 10. As such, the surgeon can precisely control retraction or dissection through small-scale and large-scale movement as endoscopic system 10 is progressively repositioned relative to the patient. The surgeon may also freely or incrementally move endoscopic system 10, or lock its position, during the procedure.

In use, a surgeon could initially position endoscopic system 10 relative to the patient. Once suitably positioned, platform 50 could be locked in a first position relative to the patient by, for example, mounting system 60. Then, the surgeon could operate slide assembly 70 to precisely move guide tube 20 relative to the patient and mounting system 60 from the first position to a second position superior to the first position. If necessary, endoscopic system 10 could be re-locked at the superior second position. The surgeon may be able to take his/her hands off one or more components of endoscopic system 10 to reposition another component or perform another task. For example, with guide tube 20 temporarily locked, bleeding vessels may be ligated or fine tissue dissection may be conducted using one or more instruments 30.

Such small-scale, large-scale, free, locked, or incremental control over the movement of endoscopic system 10 can be used to manipulate tissue generally not requiring fine dissection. This can include subcutaneous tissue located anterior to sternum 240 or manubrium 250. In general, instruments 30 can be used to perform fine dissection of, for example, nerves, vessels, or lymphatic ducts. Abdominal, thoracic, or cervical tissue may be retracted or dissected using a combination of guide tube 20 and instrument 30.

As described above, one or more different types of instruments 30 can be used in conjunction with endoscopic system 10. For example, a dissector and an electrocautery device can be used to dissect tissue and seal unwanted perforations. Alternatively, or in combination, a balloon dissector or other type of dissector may be used to create space between adjacent organs.

Insufflation may also be required to create or assist with creation of the pathway. Various parts of subcutaneous tissue may require insufflation, such as, part of the pathway encompassing the suprasternal region. Insufflation can include providing a pressurized gas, such as, for example, carbon dioxide at a pressure of 5 to 15 mmHg. Insufflation gas can be provided to a surgical site via guide tube 20 and port 18.

Once endoscopic system 10 has been navigated over sternum 240 and manubrium 250 to a location near thyroid 100 (Step 340), optical device 40 can be positioned to visualize thyroid 100. At this stage of the procedure, one or more instruments 30 may be located within guide tube 20 (Step 350). As explained above, one or more instruments 30 or optical device 40 can also be located within guide tube 20 during a prior step.

Instrument 30 can then be used to create anterior access to thyroid 100 (Step 360). This can include retracting or dissecting various organs located anterior to thyroid 100. For example, instrument 30 can be passed between a left sternothyroid muscle and a right sternothyroid muscle. Unlike prior art approaches, such retraction can be performed without penetrating a pectoralis major muscle or other major cervical and thoracic muscles.

It is possible that one or more cervical organs may require retraction during the procedures describe herein. Retraction may be necessary to access or visualize thyroid 100. For example, a Veress needle or similar device may be introduced through a lateral region of the neck to provide suitable retraction. Various other retraction techniques or devices could also be used.

In other embodiments, endoscopic system 10 could be used alone to retract one or more organs of the patient. For example, guide tube 20 or instrument 30 may be used in part to retract one or more organs. Because of their flexibility and ability to articulate, guide tube 20 or instrument 30 may be navigated around one or more organs. During such an approach, organs adjacent to thyroid 100 may be retracted via appropriate manipulation of guide tube 20 alone, or in combination with instrument 30 configured to expand or retract tissue.

To assist organ retraction or confirm sufficient access to thyroid 100, one or more optical devices 40 may be used. As explained above, optical device 40 can include a traditional endoscope, laparoscopic imaging device, or various other types of optical systems. For example, a traditional laparoscopic imaging device (not shown) may be inserted through a cervical or thoracic puncture and positioned to view thyroid 100. Alternatively, or in combination with the laparoscopic imaging device, one or more optical devices 40 may be located within guide tube 20 and positioned to view thyroid 100 or another organ near thyroid 100. As such, various imaging systems or modalities could be used with the procedures described herein. Moreover, different imaging systems or modalities could be used at different times throughout the medical procedures described herein.

In general, one or more instruments 30 can be used to divide muscles, nerves, vessels, or other organs adjacent to thyroid 100. Guide tube 20 may be articulated to provide sufficient access to a region near thyroid 100. In addition, instrument 30 or optical device 40 may be articulated or repositioned to better access regions of tissue requiring retraction or dissection. The aim of these steps is to create anterior access to thyroid 100.

The present system can also maintain improved visualization of target organs compared with traditional techniques. Visualization can be difficult to maintain with traditional devices due to their inflexibility, lack of appropriate articulation, or other limitations. In contrast, endoscopic system 10 can provide improved flexibility and articulation to maintain sufficient visualization of the target organs. For example, endoscopic system 10 can be independently operated while insufflation or retraction is maintained using other devices. Decoupling instrument control from other devices used to maintain an operating space can free a surgeon to focus more on the tissue manipulation required, rather than maintaining tissue access. As such, endoscopic system 10 can accommodate a team of surgeons, increasing the speed, accuracy, or precision of a surgical procedure.

Once thyroid 100 has been sufficiently exposed, one or more components of endoscopic system 10 may be used to dissect at least part of thyroid 100 (Step 370). Endoscopic system 10 can permit significant improvement in performing dissections of thyroid 100 because of improvements in visualization, ergonomics, and fine dissection offered by instruments 30. For example, independent articulation of optical device 40 and instruments 30 provides improved triangulation of the thyroid region as well as essential traction-countertraction. Thyroid 100 can be at least partially dissected while substantially preserving parathyroid glands 160. And unlike previous techniques, left lobe 102 and right lobe 104 can be equally accessed using a single pathway. Consequently, endoscopic system 10 permits more symmetrical visualization, access, and dissection of both lobes 102, 104.

In some embodiments, two instruments 30 can be used for dissection of thyroid 100. Each instrument 30 can be independently positioned to access either side of lobes 102, 104 from an anterior approach. Moreover, each instrument 30 can readily manipulate either lobe 102, 104 with equal dexterity and control. Various different types of instruments 30 are easily interchangeable to perform fine dissection and precisely cut, separate, ablate, or remove tissue from thyroid 100.

Tissue surrounding thyroid 100 can be viewed at various angles and examined to determine if it should be removed or left intact. Blood vessels can be selectively ligated or ablated as required. Dissection using endoscopic system 10 can allow close control of vascular pedicles, preservation of parathyroid glands 160, and ready identification of the recurrent laryngeal nerve.

After thyroid 100 has been sufficiently dissected, guide tube 20 and instruments 30 may be removed from the patient (Step 380) along with the surgical specimen. The procedures described herein generally require minimal suturing or closure. Following removal of endoscopic system 10, the incision may be closed (Step 390).

Various other surgical steps may be required during the procedures described herein or similar operations. For example, division of major blood supply may be needed to reduce blood loss during an operation. Clips, ablative energy, ligation, or other devices or methods may be used to limit unwanted bleeding. Various imaging, space creation, closure, or other steps may also be needed based on the type of procedure.

It will be apparent to those skilled in the art that various modifications and variations can be made in the disclosed devices and methods without departing from the scope of the invention. Other procedures could be performed based on subcutaneous or suprasternal access to cervical tissue. For example, parathyroid, lymph node, or carotid artery surgeries could be performed similar to the thyroid surgeries described above. In general, any anterior neck or cervical organs could be treated using endoscopic system 10. Other embodiments of the invention will be apparent to those skilled in the art from consideration of the specification and practice of the invention disclosed herein. It is intended that the specification and examples be considered as exemplary only.

What is claimed is:

1. A method of performing a medical procedure in a patient, the method comprising:
creating an incision in the patient;
inserting a flexible guide tube into the incision;
locating a flexible instrument and a flexible optical device within the guide tube;
tunneling the distal tip of the guide tube through the patient to a sternal notch or a position proximate the sternal notch; and
dissecting at least part of a thyroid using the instrument.

2. The method of claim 1, wherein the incision is in the umbilical region.

3. The method of claim 1, wherein the incision is sub-xiphoid.

4. The method of claim 1, wherein the incision is between a left mid-clavicular line and a right mid-clavicular line.

5. The method of claim 1, further including introducing a retractor through a lateral region of the neck.

6. The method of claim 1, further including positioning a distal tip of the guide tube in a suprasternal space.

7. The method of claim 6, further including insufflating the suprasternal space.

8. The method of claim 1, wherein dissecting the at least part of the thyroid includes preserving a parathyroid gland.

9. The method of claim 1, wherein dissecting the at least part of the thyroid includes superior to inferior dissection of the at least part of the thyroid.

10. The method of claim 1, further including passing the instrument between a left sternothyroid muscle and a right sternothyroid muscle without penetrating a pectoralis major muscle.

11. The method of claim 1, further including articulating the instrument and the optical device.

12. A method of performing a medical procedure in a patient, the method comprising:
creating a sub-xiphoid incision in the patient;
passing a distal tip of a flexible guide tube through the incision;
positioning the distal tip of the guide tube in a lower subcutaneous location;
tunneling the distal tip of the guide tube from the lower subcutaneous location to an upper subcutaneous location;
advancing a first flexible instrument and a second flexible instrument through the guide tube; and
dissecting at least part of a thyroid using the first instrument and the second instrument.

13. The method of claim 12, further including locating an optical device within the guide tube.

14. The method of claim 12, wherein the incision is a single incision.

15. The method of claim 12, further including positioning a platform relative to the patient, wherein the platform is fixedly positioned relative to the patient, fixedly coupled to the guide tube, and configured to permit selective movement of the guide tube relative to the patient.

16. The method of claim 15, wherein the platform is moveably coupled to the first instrument, moveably coupled to the second instrument, and configured to permit independent movement of the first instrument and the second instrument relative to the patient.

17. A method of performing a medical procedure in a patient, the method comprising:
creating an incision in an umbilicus of the patient;
passing a distal end of a flexible guide tube and a distal end of a flexible instrument through the incision;
creating a pathway from the incision to a thyroid using at least one of the guide tube and the instrument, wherein a part of the pathway is subcutaneous;
positioning an optical device to visualize the thyroid; and
dissecting at least part of the thyroid from surrounding tissue using the instrument.

18. The method of claim 17, further including insufflating at least part of the pathway.

19. The method of claim 17, further including retracting an infrahyoid muscle.

20. The method of claim 17, wherein the suprasternal part of the pathway is along a generally mid-sternal trajectory.
